(19) United States
(12) Patent Application Publication
(10) Pub. No.: US 2021/0161973 A1
(43) Pub. Date: Jun. 3, 2021

(54) AQUEOUS EXTRACT OF GROUND MATERIAL OF PLANARIAN ORGANISMS

(71) Applicants: FONDATION MEDITERRANEE INFECTION, Marseille (FR); UNIVERSITE D'AIX MARSEILLE, Marseille Cedex 7 (FR); ASSISTANCE PUBLIQUE - HOPITAUX DE MARSEILLE, Marseille (FR); CENTRE NATIONAL DE LA RECHERCHE SCIENTIFIQUE (CNRS), Paris (FR); INSERM (INSTITUT NATIONAL DE LA SANTE ET DE LA RECHERCHE MEDICALE), Paris (FR)

(72) Inventors: Eric GHIGO, Marseille (FR); Eric CHABRIERE, Aubagne (FR); Michel DRANCOURT, Marseille (FR); Vincent BONNIOL, Marseille (FR)

(73) Assignees: FONDATION MEDITERRANEE INFECTION, Marseille (FR); UNIVERSITE D'AIX MARSEILLE, Marseille Cedex 7 (FR); ASSISTANCE PUBLIQUE - HOPITAUX DE MARSEILLE, Marseille (FR); CENTRE NATIONAL DE LA RECHERCHE SCIENTIFIQUE (CNRS), Paris (FR); INSERM (INSTITUT NATIONAL DE LA SANTE ET DE LA RECHERCHE MEDICALE), Paris (FR)

(21) Appl. No.: 16/632,652

(22) PCT Filed: May 30, 2018
(86) PCT No.: PCT/FR2018/051246

§ 371 Date: Jan. 21, 2020

(30) Foreign Application Priority Data
Jul. 21, 2017 (FR) 1756929

(51) Int. Cl. A61K 35/62; C12N 5/00; A61K 8/98; A61Q 19/08; C12N 5/0775; A23L 33/10; A23L 33/00

(52) U.S. Cl. CPC A61K 35/62; C12N 5/0018; A61K 8/987; A61Q 19/08; A23V 2002/00; A23L 33/10; A23L 33/40; C12N 2500/82; C12N 5/0664

(57) ABSTRACT

The present invention relates to an aqueous extract useful notably for the regeneration of human or animal cells, notably mammalian cells, comprising an aqueous extract free of any insoluble solid debris of ground planarian organisms having a regeneration capacity, containing at least the intracellular components of the cells of said organisms of a cell lysate of said cells of said organisms.

The present invention relates to a preparation based on an extract of Platyhelminthes organisms useful notably for the regeneration of human or mammalian animal cells.

The term “planarian” is used to refer to a number of flatworms of the phylum Platyhelminthes. It mainly refers to non-exclusively parasitic freshwater Turbellaria, i.e. Paludicola, but is also used for marine Turbellaria and even for all flatworms. The Platyhelminthes are flatworms of which many species are parasites. This phylum consists mainly of worms that are elongated animals without appendages. The best known worms of the class Turbellaria (flat worms that are not exclusively parasitic) are the planarians, worms whose body thickness can measure less than one millimetre. Planarians are aquatic flatworms belonging to several species of the class Turbellaria. Planarians can swim or crawl, and live in the sea or freshwater, or in very wet soil (in tropical forests).

Planarians are organisms capable of perpetual regeneration, and any part of their body, due to the presence of a large quantity of stem cells (20-30%) in their tissues in comparison with other living organisms, which have roughly 1 ppm stem cells. Planarian stem cells multiply and regenerate continuously, unlike stem cells in more evolved organisms, which multiply little if at all. The biochemical factors that allow stem cells to multiply in a planarian cell remain unidentified.

Because of its regenerative capacity, if one part of a planarian is separated from the rest of its body, the animal recreates the other missing part in its entirety. This phenomenon of “transverse fission”, also involved in asexual reproduction, exists in other animals, particularly metazoans, but few can compete with planarians in this area.

In the article by Bocchinfuso et al. in Molecular & Cellular Proteomics vol 11 no 9, September 2012, pages 681 to 691, a non-aqueous protein extract obtained by chemical lysis of mucous secretions of Schmidtea mediterranea worms is described. In this article, only the treatment of infection and mucus disorders such as asthma and lung disease or cystic fibrosis is considered.

In the article by Changxin Ma et al in Journal of Genetics and Genomics vol 37 no 11 of November 2010, pages 713-723, here again a non-aqueous protein extract from the planarian species Dugesia japonica is described. These organic extracts are prepared for the sole purpose of proteomic study and identification of a gene involved in optic nerve regeneration.

In WO 99/33479, the therapeutic use for the treatment of autoimmune diseases of preparations based on cercariae, pathogenic parasites naturally colonizing humans and not exhibiting the property of regenerating, is described, the preparations being obtained by recovering parasite eggs from hamster liver to which said cercariae have been injected.

In the article in Hydrobiologia vol 305 no 1-3 of June 1995, pages 247-253, an extract of Dugesia japonica obtained by recovering only the heads of the Dugesia japonica organisms concerned subjected to chemical and sonication treatments and then dialysis to recover molecular extracts with a molecular size of less than 10,000 is described. In the end, only precipitated proteins are recovered. On the other hand, cell growth tests were performed on Neuro 2a and PC-12 cells. The reported results conclude that these protein extracts inhibit the growth of nerve cells and even inhibit the regeneration of the heads of said decapitated planarian organisms, whereas in the absence of treatment with these ASP planarian extracts regeneration is observed. It has been discovered, according to the present invention, that an aqueous extract of planarian flatworms, which are non-parasitic and non-pathogenic in humans or animals, notably of the species Schmidtea mediterranea, is capable of activating the multiplication of human stem cells, which planarian extract, because of this cell regeneration activity, can therefore be useful in many cosmetic or medical applications, in particular dermatological or food applications.

The expression “non-parasitic and non-pathogenic in humans or animals”, as used herein, means that such organisms do not colonize humans or mammalian animals either by ingestion or through the skin, either actively or passively, by pathologically altering human or mammalian animal cells or tissues.

More precisely, the present invention provides an aqueous extract useful notably for the regeneration of human or mammalian animal cells, comprising an aqueous extract free of any insoluble solid debris of ground organisms of the Platyhelminthes type having a regenerative capacity, said organisms being aquatic flatworms of the planarian type which are non-parasitic and non-pathogenic in humans or mammalian animals, said aqueous extract containing at least the intracellular components of the cells of said organisms of a cell lysate of said cells of said whole organisms.

The term “cell lysate”, as used herein, means that the cells of said organisms are broken down mechanically so that intracellular components are released into said extract when said organism is ground. And, said aqueous extract of ground material is an aqueous extract of said ground organisms free of any insoluble solid debris, i.e., only retained is the “planarian liquor” resulting from mechanical grinding containing only the intracellular components of the cells of said organisms of a cell lysate of said cells of said organisms, but all components and not only peptides and proteins.

More particularly, said planarian type aquatic flatworms that are non-parasitic and non-pathogenic in humans include Schmidtea mediterranea, Dugesia japonica, Dendricoelum lacteum, Polycelis nigra, Polycelys tenuis or Planana torva.

More particularly still, said aqueous extract of ground material comprises a centrifugation supernatant of said ground material.

More particularly still, said aqueous extract of ground material further comprises a cell culture medium, preferably a culture medium capable of culturing human or animal cells to be regenerated.

More particularly still, said aqueous extract of ground material comprises the grinding of 50 to 200 mg worms/mL aqueous solution, in particular for worms of 1 to 5 mg, said aqueous extract of ground material being further diluted preferably more than 1:5 and less than 1:100, preferably diluted 1:10 to 1:50; notably 1:20, preferably still diluted in said culture medium of said cells.

In particular, for worms of 2.2 mg on average, said aqueous extract of ground material comprises the ground material of 88 to 110 mg worms/mL aqueous solution, i.e. 40 to 50 worms/mL, said aqueous extract of ground material being further diluted 1:20.

More particularly still, a spectrum of said aqueous extract of ground material by MALDI-TOF mass spectrometry comprises the 16 majority characteristic peaks (i.e. higher intensities(maximum S/N value) mentioned in the following Table 1, in which the various characteristic peaks are numbered in the first column, and the following parameters have the following meanings:

M/z: ratios (mass in daltons/charge) on the x-axis of the different characteristic peaks of the spectrum,

S/N: ratios (peak intensity/background noise intensity) of the different characteristic peaks of the spectrum,

TABLE 1

| | | M/Z | | S/N |
|---|---|---|---|---|
| | | standard | | standard |
| | M/Z | deviation | S/N | deviation |
| 1 | 8618.401 | 118.031 | 10852424.610 | 11358052.088 |
| 2 | 8270.340 | 143.008 | 4620996.274 | 5564954.057 |
| 3 | 7696.982 | 133.512 | 5240359.923 | 7010776.551 |
| 4 | 7236.251 | 151.442 | 8121705.361 | 11556323.264 |
| 5 | 6803.343 | 116.896 | 10708147.034 | 18373045.879 |
| 6 | 6261.480 | 163.376 | 11319617.938 | 13604702.931 |
| 7 | 5790.573 | 116.345 | 21402383.693 | 37624416.818 |
| 8 | 5330.950 | 180.245 | 23085485.049 | 21438811.717 |
| 9 | 4787.963 | 120.365 | 22731248.668 | 23171439.149 |
| 10 | 4298.636 | 175.285 | 24470357.501 | 21953618.321 |
| 11 | 3793.559 | 113.425 | 21664847.045 | 17673326.341 |
| 12 | 3282.861 | 172.508 | 24634530.736 | 27003268.283 |
| 13 | 2792.441 | 122.205 | 22551669.576 | 22432014.766 |
| 14 | 2294.281 | 174.311 | 20589324.299 | 17568023.101 |
| 15 | 1807.544 | 115.976 | 19890585.114 | 18182405.582 |
| 16 | 1395.545 | 119.208 | 33019669.177 | 29193485.572 |

The present invention also provides a lyophilisate of an aqueous extract of ground material according to the invention.

The present invention also provides a process for producing an aqueous extract of ground material according to the invention characterized in that the steps are carried out in which:

a) said living organisms, preferably which have been washed with water and have not been fed for at least one week, are placed in an aqueous liquid medium containing preferably a cell culture medium;

b) the aqueous preparation obtained in step a) is ground, notably by mechanical grinding, until lysis of the cells of said organisms is obtained, preferably the ground material debris being between 5 and 10 μm in size (i.e. debris smaller than the size of the cells of said organisms);

c) insoluble debris is removed by centrifugation; and the supernatant is collected,

d) preferably, the centrifugation supernatant is diluted in an aqueous solution more than 1:5 and less than 1:100, preferably diluted between 1:10 and 1:50, said solution comprising also preferably components of a cell culture medium capable of culturing human or animal cells to be regenerated.

The present invention also relates to the use of an aqueous extract of ground material or lyophilisate according to the invention for the manufacture of a composition useful for regenerating human or mammalian animal cells, notably stem or differentiated cells.

In particular, mention may be made of differentiated cells of the following types: epithelial, fibroblast, keratinocyte, endothelial, neuronal or nerve.

The present invention also relates to the use of an aqueous extract of ground material or lyophilisate according to the invention for the manufacture of a composition useful for regenerating human or mammalian animal stem cells, for example a composition useful for regenerating human dental stem cells.

The present invention also relates to the use of an aqueous extract of ground material or lyophilisate according to the invention for the manufacture of a cosmetic or dermatological composition in combination with suitable carriers and/or excipients, notably an anti-ageing cosmetic or dermatological composition formulated for topical application in the form of gel, lotion (notably an aqueous solution or serum), cream, ointment, soap and paste (notably for a mask).

The present invention also relates to the use of an aqueous extract of ground material or lyophilisate according to the invention for the manufacture of a food composition.

More particularly still, an anti-ageing cosmetic product according to the invention is formulated for topical application in the form of gel, lotion, cream, ointment, soap or paste.

The topical compositions according to the invention may comprise various usual excipients suitable for external topical administration, in particular dermatologically and cosmetically acceptable excipients. These excipients suitable for formulation are well known to the skilled person and include, for example, penetrants; moisturizers; thickeners; emollients and surfactants; emulsifiers; preservatives.

These forms of topical administration are prepared by known techniques, and for example, in the case of a cream, by dispersing a fatty phase in an aqueous phase to obtain an oil-in-water emulsion, or conversely to prepare a water-in-oil emulsion.

In a particular embodiment, the cosmetic product according to the invention is formulated in the form of cream comprising, said aqueous extract and at least the following additional excipients: emulsifier, emollient, thickener, moisturizer and preservatives and pH adjuster.

In a particular embodiment, the cosmetic product according to the invention is formulated in the form of aqueous solution (notably physiological water serum) or of aqueous gel comprising, as a homogeneous mixture in water, said aqueous extract and, for the gel, at least the following additional gel excipients: gelling agent, thickener, moisturizer and preservative and pH adjuster.

The preparations generally contain other excipients and/or auxiliaries and sometimes other active principles, e.g. dyes, colouring pigments, radical scavengers, fragrances, stabilizers.

The cosmetic products may also include auxiliaries and sometimes other active principles, for example antioxidant vitamins such as vitamin E, vitamin C, antioxidant agents such as natural polyphenols, enzymes, plant active principles, natural anti-inflammatory substances, alcohols, polyols, esters, electrolytes, polar and non-polar oils, polymers, copolymers, phospholipids, dyes; fragrances; or skin scrubs.

The invention further relates to a cosmetic skin treatment process for combating the signs of skin ageing, and in particular expression wrinkles caused by uncontrolled facial muscle contractions, consisting in applying to the areas of the skin requiring such treatment, a topical cosmetic product according to the invention.

The present invention therefore relates to a cosmetic composition with an anti-ageing effect on the skin comprising an aqueous or lyophilized extract according to the invention and excipients suitable for topical application to the skin in the form of gel, lotion, cream, ointment or soap.

Other features and advantages of the present invention will become clearer by reading the description of the examples that follow, which is illustrative and non-limiting, with reference to the following figures.

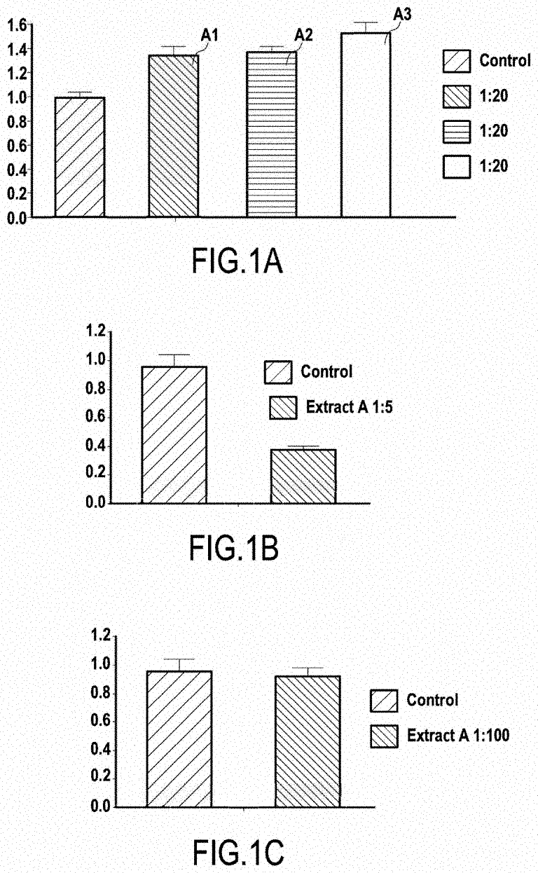

FIGS. 1A to and 1C represent the growth on day 9 of human dental stem cells treated with planarian extracts from Example 3A diluted 1:20 (FIG. 1A: aqueous extract (A1), extract containing PBS (A2) and extracts containing cell culture medium (A3) diluted 1:5 (FIG. 1B) and 1:100 (FIG. 1C).

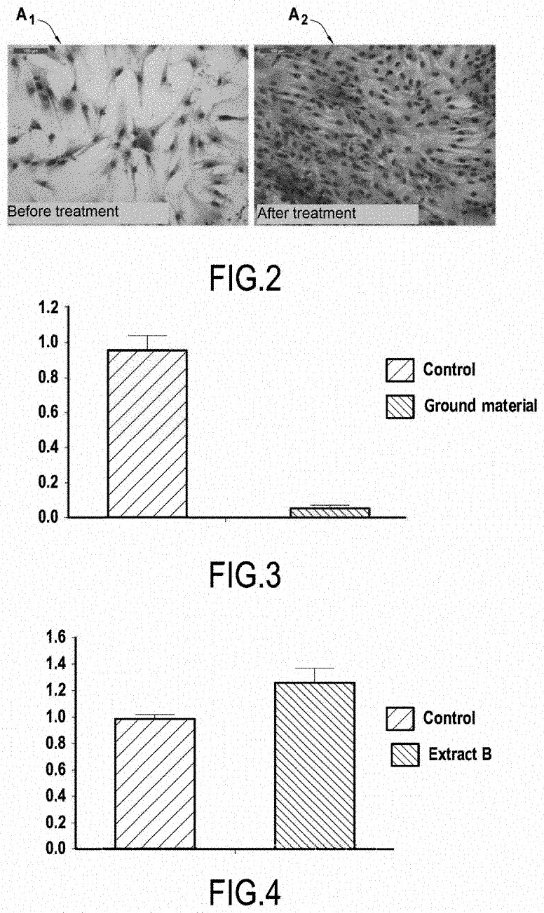

FIG. 2 represents microphotographs showing the growth of human stem cells treated with extract A diluted 1:20 for 9 days (B2) and before treatment (B1). The cells are stained with Giemsa.

FIG. 3 shows the lack of growth on day 9 of human cells treated with ground whole planarians not free of solid debris of Example 1.

FIG. 4 shows the growth of human cells treated with the solubilized lyophilized extract of Example 3B.

### 1—METHODS

1.1 Planarians:

Planarians of the species Schmidtea mediterranea are kept at 19° C. in sterile water filtered on activated carbon/ceramic cartridge and on 0.22 μm Millipore filter membrane, without antibiotics. The water is changed every two to three days in order to eliminate waste such as planarian waste. The planarians are fed calf liver once a week. They are deprived of food 1 week before the experiments to purge them. Their average weight is 2.2 mg/worm.

1.2 Stem Cells:

Human dental pulp stem cells (CLS 300702) are maintained under standard in vitro culture conditions in DMEM/F12 (GibCoBRL) cell culture medium supplemented with foetal calf serum (10%) and antibiotic compounds such as penicillin and streptomycin. (5000 U/mL, 5000 μg/mL respectively), at a temperature of 37° C. under a 5% CO₂ atmosphere. The cells are split once a week, after trypsinization (0.05%) to loosen the adherent cells, by a 1:3 dilution in DMEM/F12 culture medium (GibCoBRL) supplemented with foetal calf serum (10%) and an antibiotic cocktail (penicillin-streptomycin), gentamicin, vancomycin, ciprofloxacin) (5000 U/mL, 5000 μg/mL, 50 μg/mL, 10 μg/mL, 20 μg/mL respectively), after treatment with trypsin 0.05%, (GibCoBRL).

### EXAMPLE 1

### Preparation of the Planarian Extract (Protocol A)

1a) The planarians (25 individuals) are taken and washed twice with PBS (phosphate buffered saline, 1×) at pH=7.2, at room temperature, then placed in 550 μL of water (extract A1) or PBS (extract A2) or DMEM/F12 (GibCoBRL) culture medium (extract A3), at a concentration of 110 mg planarians/mL or 50 planarians/mL, at 4° C.

The planarians are ground with a plastic pestle, then the extract is passed 10 times (in and out) through a 23 G syringe to break up the cells, the ground debris being between 5 and 10 μm in size.

1b) The extract is then centrifuged at 200 g for 5 minutes, at a temperature of 4° C.

The centrifuged extract then has two phases, a dense phase at the bottom of the tube and a supernatant aqueous phase. The supernatant aqueous phase is then collected (about 500 μL) and stored frozen at a temperature of −80° C., or frozen in liquid nitrogen (−196° C.), as a 100 μL aliquot before use.

### EXAMPLE 2

### Preparation of Lyophilized Planarian Extract (Protocol B)

An aqueous extract of centrifugation supernatant of ground planarians containing culture medium, frozen are prepared according to protocol A above, is placed in a 2 mL glass lyophilization flask with stopper placed at a temperature of −20° C. overnight before lyophilization. Lyophilization is carried out in a COSMOS freeze-dryer from the company CRYOTEC (France) overnight. The lyophilisate is then stored at +4° C. in the dark until use. Before use the lyophilisate is resuspended in 500 μL sterile distilled water.

2. Cell Regeneration Results.

### EXAMPLE 3A

### Incubation of Human Stem Cells with the Aqueous Extract Obtained According to Protocol A

Centrifugation supernatants of ground planarians of the species S. mediterranea obtained in step 1b) of Example 1 are added at different final dilutions of 1:5, 1:20 and 1:100 to human dental stem cells at 5×10³ cells/well, of a 48-well plate, cultured in DMEM/F12 medium (GibCoBRL) supplemented with foetal calf serum (10%) and penicillin and streptomycin (5000 U/mL, 5000 μg/mL respectively), at 37° C. and under 5% CO₂ atmosphere. The cells are then maintained under the following conditions: 37° C., 5% CO₂ atmosphere for 9 days. After 9 days the cells are counted and the growth determined.

FIGS. 1A to 1C show the growth of human stem cells treated with extracts A1 to A3 diluted 1:5 (FIG. 1A), extract A3 (with culture medium) diluted 1:20 (FIG. 1B), and extract A3 (with culture medium) diluted 1:100 (FIG. 1C), for 9 days. (*p<0.05). The relative cell multiplication ratio was quantified on the y-axis compared with a control with no extract of ground material.

FIG. 2 shows the growth of human stem cells treated with extract A containing culture medium (A3) diluted 1:20 for 9 days (A2) and control before treatment (A1). The cells are stained with Giemsa.

A maximum increase with more than 50% increase in the number of cells is observed in the presence of extract A3 containing culture medium diluted 1:20 compared with the control culture without extract according to the invention, whereas for the 1:5 and 1:100 dilutions the growth is less than or equal to the control.

A maximum increase with more than 40% increase in the number of cells is observed in the presence of the aqueous extracts with water alone (A1) and containing PBS (A2) diluted 1:20 compared with the culture control without extract according to the invention.

On the other hand, FIG. 3 shows the absence of growth on day 9 of human cells treated with uncentrifuged ground whole planarians, i.e. not free of solid debris obtained in Example 1a).

### EXAMPLE 3B

### Incubation of Human Stem Cells with Lyophilized Planarian Extract (According to Protocol B)

The lyophilisate (B) is resuspended in 500 μL of water, then added at a final dilution of 1:20 to human dental stem cells at 5×10³ cells/well, of a 48-well plate, in culture in DMEM/F12 medium (GibCoBRL) supplemented with foetal calf serum (10%) and penicillin and streptomycin (5000 U/mL, 5000 μg/mL respectively), at 37° C. and under 5% CO₂ atmosphere. The cells are then maintained under the following conditions: 37° C., 5% CO₂ atmosphere for 9 days.

After 9 days the cells are counted and the growth determined. After 9 days, an increase in the number of cells in the presence of extract B of about 27.7% is observed as shown in FIG. 4.

FIG. 4 shows the growth of human stem cells treated with extract B diluted 1:20 for 9 days. (*p<0.05) The relative cell multiplication ratio was quantified on the y-axis compared with a control without extract of ground material.

### EXAMPLE 4

### MALDI-TOF Mass Spectrum of the Planarian Extract

Mass spectrometry is a known physical analytical technique for detecting and identifying molecules of interest by measuring their mass. Its principle lies in the gas phase separation of charged molecules (ions) according to their mass/charge ratio (m/z). The applications of mass spectrometry are very broad and mainly concern the identification of peptides or proteins, the analysis of their amino acid sequence or the detection of post-translational modifications.

Mass spectrometry in its MALDI-TOF (matrix-assisted laser desorption ionization-time-of-flight) variant has established itself in biological laboratories in recent years. Indeed, its advantages are numerous: (1) speed of identification (in a few minutes); (2) accuracy of identification (without the need to know the type of microorganism sought); and (3) low cost per sample (less than 50 cents per sample).

Among the mass spectrometer manufacturers, two complete MALDI-TOF systems are available for sale, marketed by Bruker Daltonics (Germany).

1. A sample of aqueous extract with water alone A1 diluted 1:20 of Example 3A was deposited on a HCCA matrix, trichloroacetic acid, water, and acetonitrile. The analysis is performed on a Microflex LT10 MALDI-TOF mass spectrometer. The results are collected with the MALDI Biotyper RTC software.

One μL of u supernatant is deposited on the target plate of the spectrometer and dried for 10 minutes. One μL of saturated HCCA matrix solution is added on top, dried for 10 minutes, then a mass spectrum of the protein extract is made on a Microflex™ LT (Bruker).

2. The spectrum acquisition method includes the following steps and features:

introduce the plate (Microflex target reference model 224989 known as MSP 96) containing the deposits into the Microflex LT mass spectrometer,

start the computer associated with the spectrometer to start the analyses, and

launch the acquisition of the MALDI-TOF mass spectra.

The parameters applied to the mass spectrometer are as follows:

positive linear mode, Electrode IS1: 20.00 kV, Electrode IS2: 18.05 kV, Focusing electrode: 6 kV, Laser frequency: 60 Hz, Detector gain: 8.8×, Post-ion-extraction (PIE): 120 ns, Mass range: 700 to 20000 Da.

The spectra are obtained by an automatic method controlled by the FLEXCONTROL® software from the manufacturer BRUKER DALTONICS® and the parameters for each spot are as follows:

serial numbers of 40 shots: 6 on different positions of the spot (6×40=240 spectra),

so that the shots have scanned the whole sample homogeneously, the acquisition is made on 6 different positions according to a hexagonal geometry,

pre-shots: 10 shots at 40% of the maximum laser power are used to desalinate the sample (contamination by mineral salts),

laser power: 30% to 45% regulated via the FLEXCONTROL® software,

spectrum selection: over a mass range of 2000 to 20,000 Da.

The acquisition of the spectra is finally carried out via the BIOTYPER® software from the manufacturer BRUKER DALTONICS® which allows the automatic method described above to be applied to a series of samples taking into account the criteria mentioned above and a quality score or validation score which represents the addition of 2 scores n1+n2 (n1 being a function of the number of peaks and n2 being a function of the background signal/noise ratio):

n1 is the score assigned according to the number of characteristic peaks in the spectrum n, where: n1=0 for n≤8, n1=1 for 8<n≤27, n1=2 for 27<n≤43, n1=3 for 43<n≤78, n1=4 for 78<n≤86, n1=5 for 86<n≤110, n1=6 for 110<n, and

n2 is the score assigned according to the value of the signal-to-noise ratio (Rmax) of the peak of greatest intensity in the spectrum, with n2=0 for Rmax≤6.8, n2=1 for 6.8<Rmax≤26.8, n2=2 for 26.8<Rmax≤52.6, n2=3 for 52.6<Rmax≤208.6, n2=4 for 208.2<Rmax≤398.6, n2=5 for 398.6<Rmax≤1,092.2, n2=6 for 1,092.2<Rmax.

A spectrum is taken into account if its validation score (n1+n2) is greater than 2.

The full spectrum is shown in Table 2 below, with the largest peaks given in Table 1 above.

In Table 2, the values given are average values over 3 extracts and 3 measurements per extract. “SD” is the standard deviation.

TABLE 2

| | m/z | | S/N |
|---|---|---|---|
| m/z | (SD) | S/N | (SD) |
| 8604.48 | 171.52 | 2944111.66 | 2325738.78 |
| 8558.14 | 103.75 | 3757582.77 | 2412169.59 |
| 8364.32 | 270.77 | 4299073.53 | 3953124.73 |
| 8231.72 | 370.12 | 6273650.35 | 4516995.49 |
| 8109.81 | 473.70 | 14474095.01 | 11993621.48 |
| 8091.08 | 485.10 | 16468398.81 | 10785668.45 |
| 8066.99 | 473.89 | 10995647.50 | 9275395.50 |
| 8040.23 | 453.94 | 5195376.26 | 413314.92 |
| 8028.26 | 448.60 | 1362266.53 | 564362.67 |
| 8007.05 | 458.07 | 1529078.69 | 723901.78 |
| 7977.47 | 449.60 | 1934246.85 | 606979.25 |
| 7956.26 | 430.41 | 3779700.78 | 2089476.26 |
| 7926.83 | 439.17 | 6343305.77 | 2204916.89 |
| 7915.14 | 445.50 | 5271199.78 | 3945913.58 |
| 7888.82 | 422.60 | 9915944.44 | 6394858.15 |
| 7845.95 | 451.04 | 8949566.35 | 8892715.74 |
| 7774.23 | 390.89 | 10047314.25 | 14751532.02 |
| 7766.87 | 396.60 | 17998107.53 | 16324726.64 |
| 7739.54 | 417.36 | 6983093.88 | 4683065.73 |
| 7711.45 | 420.11 | 11638867.28 | 11162939.84 |
| 7682.37 | 447.41 | 2849076.46 | 26573.96 |
| 7620.78 | 384.09 | 1522404.95 | 1571602.58 |
| 7603.31 | 366.14 | 3797954.75 | 301700.94 |
| 7507.22 | 462.35 | 4988724.05 | 1053369.21 |
| 7460.86 | 447.47 | 3010910.15 | 786290.91 |
| 7453.34 | 443.82 | 1473989.18 | 754971.29 |
| 7441.65 | 442.85 | 2268420.85 | 265803.84 |
| 7428.84 | 436.23 | 990780.63 | 482569.27 |
| 7411.46 | 434.77 | 3113006.65 | 44381.36 |
| 7379.53 | 413.93 | 2507654.97 | 237260.33 |
| 7357.69 | 419.02 | 3439236.21 | 1696465.36 |
| 7348.32 | 417.87 | 4953557.73 | 865045.30 |
| 7337.73 | 413.55 | 5789551.76 | 904998.52 |
| 7327.41 | 406.71 | 1938361.88 | 100617.30 |
| 7298.87 | 418.81 | 8970671.17 | 10923542.62 |
| 7291.71 | 416.32 | 15579329.31 | 11046360.56 |
| 7233.87 | 458.44 | 2108878.03 | 1112809.94 |
| 7224.58 | 467.28 | 2188902.66 | 1253603.56 |
| 7211.60 | 462.63 | 1277388.33 | 46435.77 |
| 7183.03 | 474.47 | 10858543.09 | 6841857.42 |
| 7166.12 | 461.61 | 6005883.09 | 4377180.05 |
| 7141.72 | 462.27 | 8454369.88 | 12773497.18 |
| 7127.32 | 462.44 | 15980137.91 | 13198728.24 |
| 7117.16 | 460.20 | 1943880.50 | 1297607.73 |
| 7098.11 | 474.95 | 28478914.80 | 34393160.51 |
| 7089.16 | 479.62 | 40768433.01 | 42971633.85 |
| 7068.69 | 472.09 | 3149404.57 | 1069351.59 |
| 7057.38 | 475.83 | 4015369.66 | 840859.73 |
| 7049.75 | 475.55 | 3557870.44 | 341332.63 |
| 7046.15 | 475.01 | 3404514.08 | 572811.01 |
| 7029.85 | 469.40 | 16173381.98 | 27409009.49 |
| 7019.69 | 465.68 | 29617067.96 | 31933238.72 |
| 6999.99 | 447.67 | 10403410.49 | 4241581.40 |
| 6994.31 | 450.26 | 12077581.56 | 3743384.83 |
| 6971.95 | 439.31 | 19264755.03 | 20608829.34 |
| 6958.98 | 433.46 | 7134836.63 | 5304127.76 |
| 6948.13 | 440.32 | 9541839.49 | 15202317.68 |
| 6927.62 | 429.41 | 19426439.15 | 15239899.38 |
| 6905.24 | 428.66 | 8416940.87 | 2205389.06 |
| 6896.02 | 426.82 | 5501032.42 | 1797378.54 |
| 6873.20 | 404.03 | 5662071.47 | 988813.38 |
| 6862.21 | 407.24 | 5647756.06 | 486820.14 |
| 6849.90 | 398.50 | 4232957.36 | 2380406.49 |
| 6843.00 | 401.23 | 2871706.78 | 8206.17 |
| 6831.95 | 396.09 | 11137649.21 | 7908745.51 |
| 6820.90 | 395.54 | 4224912.36 | 1165458.98 |
| 6805.35 | 398.01 | 4634248.86 | 2098831.45 |
| 6800.77 | 396.39 | 5973899.73 | 1989363.37 |
| 6782.34 | 393.37 | 19055281.15 | 21136742.86 |
| 6770.31 | 391.57 | 35509917.83 | 13034885.15 |
| 6745.15 | 407.32 | 18571000.28 | 15262519.68 |
| 6725.36 | 408.71 | 8653775.85 | 4308862.55 |
| 6709.87 | 408.66 | 5238132.91 | 1539867.36 |
| 6700.89 | 409.86 | 7534629.94 | 2915163.71 |
| 6696.13 | 411.06 | 8830295.99 | 3256750.86 |
| 6680.99 | 407.15 | 4231387.69 | 593937.32 |
| 6654.14 | 430.44 | 3521998.94 | 1238912.77 |
| 6640.58 | 422.93 | 3211633.91 | 246128.86 |
| 6625.94 | 423.15 | 1532349.37 | 792001.80 |
| 6615.85 | 426.74 | 6569850.42 | 2709202.72 |
| 6600.71 | 441.14 | 5420849.20 | 2607427.19 |
| 6581.25 | 440.63 | 4607565.27 | 5977204.13 |
| 6571.96 | 433.59 | 8440513.66 | 5900474.08 |
| 6560.55 | 441.43 | 6770162.75 | 4190931.56 |
| 6548.88 | 443.03 | 8297991.19 | 897107.33 |
| 6539.51 | 441.18 | 1698707.19 | 918513.03 |
| 6520.81 | 429.87 | 4671420.38 | 784239.83 |
| 6510.06 | 437.54 | 11852564.16 | 1975111.53 |
| 6497.56 | 430.01 | 9963548.39 | 4648988.58 |
| 6486.81 | 435.57 | 11018822.76 | 5444923.89 |
| 6478.80 | 431.50 | 10627202.29 | 7294336.61 |
| 6471.06 | 431.57 | 6997871.01 | 6763605.29 |
| 6457.19 | 441.74 | 9893297.41 | 8332244.06 |
| 6453.29 | 438.94 | 3714855.88 | 283840.79 |
| 6442.34 | 432.99 | 13686239.46 | 11824081.49 |
| 6430.23 | 428.59 | 5363664.11 | 3097124.33 |
| 6418.96 | 432.14 | 5051217.13 | 1408786.51 |
| 6406.03 | 428.78 | 4700220.72 | 4284253.70 |
| 6401.90 | 428.42 | 5939522.66 | 4703337.99 |
| 6387.38 | 431.14 | 4660595.31 | 684934.44 |
| 6376.39 | 431.18 | 4951125.32 | 1356190.69 |
| 6362.96 | 431.07 | 2672270.13 | 1511767.26 |
| 6355.85 | 431.23 | 6478221.94 | 2029795.02 |
| 6337.93 | 429.77 | 14364514.88 | 13011112.00 |
| 6334.17 | 429.91 | 23516628.10 | 10002697.44 |
| 6315.15 | 419.82 | 53493918.67 | 60231591.15 |
| 6301.95 | 415.75 | 4271746.45 | 1331550.38 |
| 6296.98 | 415.38 | 7983195.65 | 3877555.77 |
| 6289.60 | 421.89 | 4707429.99 | 374730.32 |
| 6282.32 | 424.31 | 7918858.10 | 7263748.27 |
| 6266.95 | 425.22 | 24637014.27 | 5239080.77 |
| 6256.93 | 425.36 | 20987838.34 | 1490238.07 |
| 6237.88 | 427.33 | 29937581.00 | 13197002.41 |
| 6211.75 | 416.15 | 22008681.24 | 24713621.54 |
| 6195.65 | 418.84 | 33438558.07 | 29633341.75 |
| 6182.69 | 409.79 | 15858769.83 | 1500875.04 |
| 6167.23 | 403.94 | 4936405.49 | 1627499.18 |
| 6163.90 | 404.28 | 5019684.98 | 2599607.88 |
| 6156.47 | 407.31 | 2624490.77 | 1286496.35 |
| 6147.22 | 401.26 | 7887713.01 | 12198784.12 |
| 6143.52 | 399.94 | 14105662.43 | 13934913.91 |
| 6131.16 | 394.92 | 6354446.85 | 8095590.50 |
| 6118.66 | 384.91 | 32928869.15 | 22660659.81 |
| 6108.38 | 379.45 | 33705975.23 | 37481913.75 |
| 6085.16 | 372.75 | 21732618.28 | 24358022.96 |
| 6076.11 | 369.49 | 41806833.28 | 10520817.79 |
| 6058.74 | 364.32 | 5401884.41 | 2873516.50 |
| 6053.17 | 364.09 | 5289816.75 | 4967671.09 |
| 6049.05 | 360.71 | 5039738.82 | 4484807.34 |
| 6039.69 | 359.70 | 7950100.05 | 3741188.19 |
| 6028.98 | 362.47 | 2869743.69 | 1127718.20 |
| 6022.25 | 362.63 | 6313650.43 | 3642689.26 |
| 6018.84 | 362.50 | 8471752.32 | 3281285.87 |
| 6006.88 | 359.80 | 5409871.27 | 3113330.06 |
| 5993.85 | 362.93 | 11073405.03 | 7987999.30 |
| 5989.40 | 363.15 | 14780907.47 | 8952105.05 |
| 5976.57 | 357.54 | 8220268.73 | 3780515.24 |
| 5965.81 | 359.65 | 35958240.78 | 61288743.82 |
| 5958.71 | 362.18 | 66081970.83 | 71289746.18 |
| 5949.95 | 364.41 | 11209352.45 | 2940139.47 |
| 5939.47 | 361.28 | 7387542.62 | 3210857.02 |
| 5934.00 | 358.69 | 5099206.50 | 281578.79 |
| 5927.71 | 356.18 | 5285416.84 | 2063775.29 |
| 5921.72 | 357.90 | 5141686.87 | 3613314.69 |
| 5913.57 | 352.22 | 7891140.07 | 5273019.26 |
| 5907.89 | 351.46 | 8761129.52 | 4214337.97 |
| 5897.28 | 343.10 | 16943379.29 | 13133259.91 |
| 5887.39 | 335.68 | 9519092.77 | 5864957.51 |
| 5875.52 | 339.85 | 11843506.86 | 9571102.04 |
| 5865.73 | 338.82 | 11879994.37 | 10762952.96 |
| 5852.10 | 350.61 | 3491143.90 | 815315.18 |
| 5847.66 | 347.41 | 2478062.53 | 684480.52 |
| 5839.13 | 348.83 | 11178026.11 | 8787186.69 |
| 5833.41 | 345.72 | 3786800.88 | 1356341.36 |
| 5828.30 | 349.66 | 6738523.40 | 5523587.00 |
| 5820.71 | 343.85 | 15333784.17 | 9579858.47 |
| 5812.92 | 346.25 | 16233521.23 | 12858822.74 |
| 5801.36 | 337.53 | 5914522.21 | 2838342.56 |
| 5793.34 | 335.16 | 3836244.22 | 1878433.00 |
| 5788.87 | 333.69 | 5546783.31 | 1135522.40 |
| 5779.83 | 338.17 | 3085730.60 | 467020.28 |
| 5774.44 | 339.69 | 4216817.00 | 1505309.25 |
| 5761.75 | 341.14 | 26029693.44 | 19860563.73 |
| 5745.50 | 352.24 | 15330816.80 | 403931.59 |
| 5738.63 | 352.36 | 1375856.71 | 250523.03 |
| 5728.45 | 355.77 | 21143197.37 | 13187223.44 |
| 5721.62 | 359.69 | 9986411.36 | 6367273.06 |
| 5710.31 | 351.04 | 17962783.76 | 7625526.14 |
| 5698.31 | 354.30 | 16289738.67 | 13847283.44 |
| 5692.42 | 352.19 | 8642010.82 | 2879857.07 |
| 5679.18 | 357.29 | 16949029.66 | 8469501.39 |
| 5671.41 | 354.40 | 23610643.88 | 902915.57 |
| 5659.88 | 351.82 | 10741135.91 | 10434945.84 |
| 5650.07 | 345.04 | 9719910.27 | 11106900.98 |
| 5642.02 | 346.77 | 20837828.42 | 6120062.17 |
| 5632.28 | 342.04 | 5179586.28 | 3748131.62 |
| 5625.34 | 343.43 | 11783997.01 | 14900296.16 |
| 5615.84 | 339.78 | 36318300.21 | 2666610.42 |
| 5594.82 | 346.11 | 29642064.46 | 5400857.11 |
| 5576.71 | 341.97 | 52675793.29 | 16242152.20 |
| 5562.64 | 335.33 | 44208515.45 | 14139358.56 |
| 5533.29 | 349.04 | 38001603.57 | 19382761.03 |
| 5517.52 | 350.03 | 10420201.24 | 6231351.60 |
| 5514.12 | 351.64 | 5291279.59 | 3523589.34 |
| 5509.38 | 350.32 | 12999875.87 | 11017133.88 |
| 5497.07 | 351.52 | 3561189.66 | 1240547.00 |
| 5486.71 | 346.65 | 15671783.68 | 11138988.70 |
| 5479.87 | 340.82 | 8069278.92 | 313063.01 |
| 5475.26 | 342.02 | 3440691.28 | 1935367.77 |
| 5464.95 | 341.03 | 34954370.15 | 28250458.38 |
| 5457.03 | 336.29 | 13737638.07 | 10554844.99 |
| 5453.15 | 338.55 | 6501242.18 | 3186060.96 |
| 5445.57 | 334.05 | 4466355.05 | 1814188.14 |
| 5439.27 | 333.36 | 6848806.84 | 1165890.91 |
| 5430.64 | 339.46 | 20559236.29 | 15470533.29 |
| 5417.81 | 342.77 | 15124914.03 | 8167741.94 |
| 5408.51 | 347.33 | 13648603.78 | 9704645.70 |
| 5400.13 | 347.18 | 12155209.18 | 3380351.47 |
| 5387.63 | 344.09 | 17840291.22 | 8774788.25 |
| 5382.04 | 342.69 | 23047717.18 | 9033382.84 |
| 5374.43 | 341.23 | 2463218.45 | 1607909.35 |
| 5372.03 | 340.71 | 2088536.80 | 988011.65 |
| 5361.09 | 333.67 | 123782598.90 | 121768630.37 |
| 5346.40 | 326.67 | 2170802.88 | 446965.68 |
| 5339.79 | 325.26 | 16046753.94 | 4577883.99 |
| 5330.27 | 327.57 | 10380188.29 | 3530935.54 |
| 5322.83 | 329.73 | 12972876.56 | 4355119.79 |
| 5313.58 | 329.45 | 4903376.42 | 860576.34 |
| 5303.04 | 320.68 | 18504824.66 | 16061179.49 |
| 5296.70 | 318.56 | 18484406.43 | 19494127.81 |
| 5285.43 | 318.86 | 18523147.47 | 8835491.49 |
| 5281.34 | 320.41 | 12807973.25 | 10262991.81 |
| 5273.38 | 313.19 | 6569793.87 | 7088402.52 |
| 5261.30 | 324.47 | 28666017.67 | 13429441.44 |
| 5251.70 | 326.52 | 2974198.88 | 761102.86 |
| 5244.71 | 323.47 | 4150055.83 | 1939423.97 |
| 5233.98 | 332.00 | 6627720.37 | 3606824.76 |
| 5228.24 | 331.02 | 5468854.68 | 5499954.51 |
| 5222.71 | 327.78 | 9870764.21 | 6649045.63 |
| 5216.91 | 328.50 | 11599727.97 | 6568680.89 |
| 5209.48 | 326.52 | 5873514.64 | 3082995.21 |
| 5201.13 | 320.74 | 2790965.92 | 229588.96 |
| 5195.71 | 317.84 | 35784893.28 | 39766059.29 |
| 5185.74 | 311.10 | 10482160.64 | 871436.85 |
| 5176.37 | 313.72 | 8204843.69 | 2916964.18 |
| 5170.09 | 314.46 | 14224580.66 | 4634851.87 |
| 5158.00 | 323.74 | 21682802.23 | 32411036.77 |
| 5146.29 | 320.70 | 62792385.27 | 7495217.41 |
| 5131.17 | 321.65 | 15708286.22 | 10318889.12 |
| 5118.22 | 320.49 | 13894513.60 | 7938662.73 |
| 5113.94 | 322.21 | 18650972.48 | 5802374.05 |
| 5104.87 | 323.71 | 15164468.42 | 11575196.95 |
| 5095.35 | 315.99 | 25322556.38 | 20024197.59 |
| 5081.68 | 312.64 | 17341409.81 | 13289025.37 |
| 5070.42 | 312.06 | 11172273.01 | 8222476.82 |
| 5061.87 | 310.70 | 25382672.43 | 14897323.03 |
| 5053.41 | 314.99 | 28450338.86 | 19802450.93 |
| 5040.53 | 304.64 | 26604974.51 | 13830524.23 |
| 5029.37 | 302.55 | 19143524.07 | 20449223.69 |
| 5017.45 | 296.49 | 52583858.93 | 41063311.15 |
| 5003.58 | 290.36 | 3684320.31 | 716982.52 |
| 4997.46 | 290.07 | 5761472.80 | 4205591.33 |
| 4992.06 | 292.05 | 10054343.14 | 2343055.55 |
| 4985.36 | 287.29 | 36486614.23 | 32430181.46 |
| 4967.33 | 271.87 | 24008797.78 | 24042783.06 |
| 4956.35 | 265.21 | 31323844.57 | 28074611.19 |
| 4951.60 | 264.17 | 10166089.25 | 6261355.28 |
| 4943.59 | 259.15 | 31102776.61 | 30415431.35 |
| 4924.50 | 249.73 | 29036164.87 | 1307616.44 |
| 4909.24 | 246.65 | 21453204.84 | 2012994.93 |
| 4898.38 | 240.27 | 13411158.56 | 8574930.85 |
| 4890.83 | 235.97 | 21288911.94 | 5128276.83 |
| 4884.18 | 237.62 | 15429403.74 | 14925659.38 |
| 4876.55 | 233.83 | 14297057.62 | 14803682.90 |
| 4870.39 | 234.85 | 20996189.03 | 17920300.23 |
| 4863.92 | 229.23 | 11794700.50 | 5911751.61 |
| 4849.99 | 224.42 | 33732314.61 | 22390766.55 |
| 4841.26 | 220.30 | 5305602.20 | 3589999.37 |
| 4831.21 | 212.51 | 34994332.46 | 33720525.84 |
| 4823.83 | 207.62 | 2092124.97 | 537135.20 |
| 4817.59 | 203.89 | 18707619.15 | 18154097.56 |
| 4808.41 | 198.95 | 12331367.52 | 9770015.05 |
| 4800.61 | 191.68 | 17287289.97 | 13395785.32 |
| 4789.74 | 188.89 | 10072772.38 | 12346992.07 |
| 4786.59 | 188.88 | 16625664.49 | 13743556.93 |
| 4773.69 | 180.10 | 7897514.40 | 3006202.07 |
| 4768.60 | 176.31 | 11808778.42 | 1486176.30 |
| 4751.77 | 180.55 | 21376139.50 | 12343262.67 |
| 4746.36 | 178.10 | 21195878.24 | 20892585.73 |
| 4740.11 | 174.11 | 8580533.73 | 6546440.16 |
| 4724.02 | 163.59 | 62669902.12 | 50782550.17 |
| 4711.23 | 153.15 | 28855545.77 | 11634700.57 |
| 4699.85 | 158.47 | 15135928.22 | 16045727.86 |
| 4690.74 | 158.90 | 21144355.15 | 37596339.24 |
| 4685.67 | 158.87 | 41052419.93 | 42088172.66 |
| 4676.42 | 167.18 | 14110006.36 | 10611535.52 |
| 4669.19 | 168.96 | 8988706.42 | 2837513.23 |
| 4658.16 | 160.08 | 36645769.34 | 38317167.30 |
| 4647.18 | 158.69 | 28604979.37 | 14898916.39 |
| 4637.04 | 152.88 | 19933826.17 | 21968963.92 |
| 4629.52 | 156.37 | 22604888.16 | 22309592.89 |
| 4619.74 | 149.35 | 4681308.24 | 1126018.78 |
| 4604.57 | 134.42 | 34425481.52 | 33346047.24 |
| 4596.16 | 131.37 | 37674118.04 | 38618050.32 |
| 4584.77 | 120.75 | 4338132.53 | 992300.08 |
| 4581.11 | 120.70 | 13130307.22 | 7380331.67 |
| 4573.44 | 118.66 | 8672729.08 | 2432100.64 |
| 4567.85 | 118.27 | 13550438.84 | 1535884.11 |
| 4562.05 | 115.15 | 20784474.14 | 6421894.87 |
| 4551.94 | 112.56 | 6764446.51 | 1476598.81 |
| 4548.00 | 113.30 | 2639582.16 | 745930.56 |
| 4539.59 | 115.82 | 15514582.46 | 6584376.81 |
| 4534.60 | 117.52 | 15823408.86 | 2660867.27 |
| 4525.72 | 112.17 | 12423707.56 | 14396860.07 |
| 4521.25 | 112.78 | 18904209.92 | 17450137.61 |
| 4510.81 | 107.35 | 20648226.60 | 14837677.51 |
| 4502.04 | 105.01 | 5464260.70 | 4155079.40 |
| 4489.94 | 113.88 | 23027592.44 | 21697464.34 |
| 4478.08 | 115.96 | 32809807.30 | 28786518.28 |
| 4472.46 | 111.87 | 13190484.07 | 3124588.34 |
| 4458.16 | 107.83 | 57673698.99 | 62245368.31 |
| 4451.65 | 104.32 | 3045465.26 | 644076.41 |
| 4445.75 | 100.98 | 10726190.67 | 7209722.85 |
| 4438.22 | 99.13 | 9177047.88 | 6607749.32 |
| 4432.40 | 94.23 | 22511700.94 | 21273557.90 |
| 4425.83 | 92.52 | 28030426.41 | 29655879.89 |
| 4414.73 | 87.94 | 15380252.97 | 9737228.73 |
| 4409.48 | 86.48 | 21645758.44 | 8497514.80 |
| 4400.24 | 91.42 | 11990439.24 | 9552690.64 |
| 4393.28 | 91.37 | 22750366.24 | 4094827.06 |
| 4383.76 | 94.96 | 24377855.77 | 7453548.07 |
| 4375.15 | 91.85 | 18069142.29 | 14922408.99 |
| 4361.16 | 88.90 | 38764160.29 | 21996218.44 |
| 4347.12 | 79.46 | 23062492.69 | 18863117.08 |
| 4340.05 | 80.17 | 10485992.55 | 8832780.02 |
| 4335.06 | 79.30 | 14438570.33 | 10175943.67 |
| 4326.04 | 76.61 | 40250972.75 | 19662581.27 |
| 4315.14 | 72.10 | 33715945.96 | 3980366.15 |
| 4298.40 | 74.40 | 15668005.20 | 15471228.20 |
| 4289.65 | 74.24 | 8610064.57 | 5271904.89 |
| 4280.78 | 79.99 | 29282616.75 | 17913297.45 |
| 4266.41 | 73.95 | 23544094.70 | 16612720.28 |
| 4257.35 | 73.57 | 29823758.86 | 21326317.62 |
| 4247.19 | 75.13 | 26445601.36 | 22554207.34 |
| 4239.75 | 73.05 | 46558890.10 | 24606139.74 |
| 4228.99 | 81.27 | 30750371.83 | 33572037.23 |
| 4216.43 | 68.69 | 20839097.87 | 23402073.32 |
| 4209.16 | 64.38 | 39509777.67 | 7701342.16 |
| 4193.32 | 66.02 | 21453874.22 | 15504858.45 |
| 4187.78 | 62.93 | 10221980.88 | 7242193.29 |
| 4180.66 | 64.92 | 22215591.07 | 19471886.67 |
| 4171.79 | 58.00 | 18221938.19 | 13521735.12 |
| 4161.37 | 59.73 | 30059237.97 | 10207324.43 |
| 4152.96 | 59.60 | 7764954.52 | 9278003.06 |
| 4148.18 | 60.38 | 14096581.90 | 8740782.19 |
| 4141.87 | 61.11 | 19969477.54 | 17033711.27 |
| 4138.49 | 62.69 | 33808733.57 | 8197165.45 |
| 4125.52 | 57.49 | 14230924.48 | 11146310.35 |
| 4117.36 | 53.64 | 2510469.06 | 1338616.50 |
| 4110.47 | 47.29 | 29363728.48 | 28985033.06 |
| 4095.25 | 41.10 | 17924303.60 | 16359894.32 |
| 4089.58 | 39.38 | 31900280.25 | 2189534.16 |
| 4076.74 | 42.75 | 27372504.36 | 16862469.84 |
| 4070.79 | 40.55 | 22315462.87 | 23339463.51 |
| 4061.63 | 45.74 | 6871552.62 | 1628836.56 |
| 4059.72 | 45.45 | 8231697.72 | 561907.51 |
| 4051.95 | 45.87 | 16775790.26 | 14682853.10 |
| 4043.49 | 42.30 | 18316937.80 | 6741009.85 |
| 4036.73 | 43.09 | 8888168.60 | 10919111.80 |
| 4031.86 | 41.98 | 14706107.89 | 12156976.32 |
| 4019.71 | 43.17 | 30001635.10 | 18160662.06 |
| 4011.99 | 38.67 | 12380634.05 | 5890032.36 |
| 4004.09 | 39.69 | 18799524.41 | 21337495.93 |
| 3996.81 | 43.38 | 37417358.56 | 14956624.57 |
| 3977.66 | 29.53 | 33685146.18 | 25920132.08 |
| 3971.37 | 25.42 | 12181406.39 | 6032114.93 |
| 3965.97 | 26.86 | 11790909.60 | 7959273.44 |
| 3961.78 | 23.70 | 11675027.43 | 6899033.74 |
| 3955.38 | 21.64 | 9503238.05 | 2548075.31 |
| 3944.59 | 17.11 | 40925363.15 | 8707644.10 |
| 3932.58 | 18.91 | 25949184.21 | 11860229.70 |
| 3915.27 | 18.11 | 26412628.26 | 17215837.21 |
| 3905.31 | 16.15 | 15089827.13 | 12462343.31 |
| 3900.10 | 13.98 | 22203847.90 | 12790395.82 |
| 3889.08 | 9.65 | 18495973.81 | 24340959.77 |
| 3879.43 | 5.07 | 51177131.99 | 9758836.59 |
| 3869.82 | 8.40 | 38583244.10 | 30653038.16 |
| 3860.10 | 0.80 | 18030165.78 | 5567109.91 |
| 3848.48 | 6.70 | 13855277.63 | 4316262.15 |
| 3842.37 | 4.29 | 15558318.67 | 5493452.66 |
| 3834.76 | 3.35 | 11577397.75 | 8918070.40 |
| 3829.12 | 4.75 | 23050578.64 | 7734076.75 |
| 3820.79 | 2.38 | 30841222.29 | 3141004.91 |
| 3812.19 | 7.60 | 7598710.04 | 5937924.71 |
| 3808.30 | 7.00 | 11293631.97 | 4795503.58 |
| 3801.75 | 5.27 | 21038007.51 | 15286189.62 |
| 3793.79 | 5.06 | 18961775.35 | 13751410.74 |
| 3790.47 | 5.51 | 20305269.38 | 18159323.54 |
| 3784.15 | 3.69 | 11322006.85 | 9187316.57 |
| 3780.44 | 1.05 | 19718682.86 | 6171115.73 |
| 3771.48 | 4.33 | 4604201.77 | 3334039.91 |
| 3763.07 | 6.49 | 31505321.38 | 20870410.51 |
| 3756.01 | 4.74 | 27786964.11 | 22867189.49 |
| 3748.77 | 6.34 | 4086444.48 | 1576380.13 |
| 3739.01 | 3.19 | 34198092.81 | 32052236.68 |
| 3730.76 | 9.15 | 21113246.74 | 7579176.73 |
| 3720.99 | 13.71 | 30012847.89 | 29347983.55 |
| 3708.93 | 14.18 | 12152550.72 | 8097962.82 |
| 3704.13 | 14.88 | 25989389.84 | 2416799.68 |
| 3696.44 | 17.37 | 3469796.56 | 3549517.63 |
| 3693.88 | 16.51 | 19459441.25 | 12378760.59 |
| 3681.29 | 10.06 | 27935096.29 | 7974994.36 |
| 3671.57 | 11.65 | 23609986.91 | 5175207.70 |
| 3666.47 | 14.43 | 9661023.89 | 14192233.65 |
| 3660.26 | 11.71 | 29084285.26 | 2147309.89 |
| 3653.86 | 12.77 | 5739320.30 | 4707533.46 |
| 3647.72 | 17.05 | 12419233.69 | 4457564.18 |
| 3641.61 | 16.65 | 18260770.54 | 20542984.89 |
| 3639.59 | 16.95 | 25774278.16 | 24683868.50 |
| 3623.21 | 25.82 | 26932197.35 | 27155932.40 |
| 3617.37 | 23.74 | 47999375.90 | 16186311.04 |
| 3603.60 | 27.15 | 25735041.86 | 8557697.98 |
| 3595.82 | 29.50 | 23592435.49 | 14859889.10 |
| 3585.66 | 31.83 | 15765186.17 | 7520257.83 |
| 3575.08 | 28.48 | 27036985.06 | 8799617.08 |
| 3568.70 | 27.31 | 18685579.75 | 18273339.89 |
| 3562.87 | 23.13 | 5344386.11 | 2521967.97 |
| 3558.35 | 24.37 | 20060783.74 | 16795784.20 |
| 3549.78 | 29.67 | 8202388.26 | 5002844.62 |
| 3546.10 | 28.69 | 21742082.50 | 15525320.56 |
| 3531.50 | 22.99 | 37762348.39 | 15996660.43 |
| 3523.33 | 18.38 | 17135206.56 | 9485074.69 |
| 3513.20 | 26.29 | 17775779.54 | 16510220.06 |
| 3509.71 | 26.65 | 25024640.33 | 18256091.78 |
| 3499.01 | 28.30 | 8378579.74 | 7548694.72 |
| 3496.24 | 26.38 | 12705186.15 | 7984652.76 |
| 3481.00 | 25.51 | 108990693.56 | 76439080.70 |
| 3468.85 | 31.12 | 26850794.82 | 20905162.31 |
| 3460.71 | 37.20 | 25811787.96 | 12033545.35 |
| 3449.50 | 41.78 | 15579199.82 | 3596568.74 |
| 3443.63 | 41.39 | 12420646.29 | 3293489.54 |
| 3439.39 | 39.46 | 10030046.47 | 230300.00 |
| 3435.85 | 38.87 | 10922343.50 | 1843035.38 |
| 3423.28 | 50.62 | 27944680.80 | 30333985.68 |
| 3414.98 | 47.67 | 74726957.91 | 55458172.32 |
| 3403.60 | 47.28 | 13819748.87 | 2628103.69 |
| 3397.00 | 53.49 | 20605663.45 | 18854512.12 |
| 3389.09 | 51.80 | 39127210.66 | 19805729.21 |
| 3382.65 | 54.56 | 69115539.76 | 22654554.18 |
| 3371.79 | 51.65 | 9985734.18 | 923862.05 |
| 3367.59 | 49.88 | 10387476.70 | 3668574.97 |
| 3360.88 | 50.94 | 16104356.62 | 9900405.05 |
| 3350.63 | 58.68 | 30184055.30 | 12310656.26 |
| 3345.60 | 61.05 | 14747773.25 | 15142285.91 |
| 3337.71 | 63.24 | 7664061.32 | 5845420.44 |
| 3331.66 | 58.65 | 18421246.43 | 16215909.26 |
| 3325.25 | 57.22 | 47689800.23 | 42047417.29 |
| 3312.74 | 68.83 | 24370101.45 | 9874452.44 |
| 3308.53 | 69.60 | 6106740.31 | 4913806.13 |
| 3298.20 | 67.73 | 31437156.51 | 3807777.94 |
| 3292.91 | 67.60 | 8023804.80 | 4939263.05 |
| 3287.64 | 64.69 | 21028204.39 | 18579686.40 |
| 3281.38 | 60.64 | 39552460.76 | 10292440.16 |
| 3270.41 | 61.20 | 27608241.56 | 25175542.96 |
| 3266.57 | 59.35 | 32971928.57 | 33706720.32 |
| 3254.19 | 57.99 | 34350370.49 | 22244064.03 |
| 3247.96 | 54.01 | 4762134.58 | 326311.48 |
| 3241.32 | 52.81 | 32161482.27 | 11525336.81 |
| 3233.71 | 54.58 | 17714314.79 | 16497325.56 |
| 3226.47 | 61.79 | 12597550.05 | 10527254.42 |
| 3220.27 | 61.87 | 23157503.92 | 22260709.69 |
| 3211.59 | 60.41 | 13984204.26 | 11618435.63 |
| 3204.70 | 59.28 | 25670846.92 | 16627993.03 |
| 3200.53 | 58.57 | 20927873.65 | 21034473.80 |
| 3194.07 | 54.09 | 31205066.12 | 20661811.88 |
| 3188.63 | 49.63 | 32337486.19 | 25789203.35 |
| 3174.78 | 59.59 | 18909334.60 | 13050314.39 |
| 3167.91 | 57.35 | 29177573.97 | 14227675.90 |
| 3161.99 | 58.43 | 21266164.12 | 22024188.90 |
| 3158.11 | 55.76 | 3118499.37 | 986032.68 |
| 3153.84 | 58.89 | 10676838.04 | 9693794.23 |
| 3146.48 | 61.38 | 10925248.09 | 11451894.61 |
| 3142.74 | 60.26 | 17515360.23 | 9749913.47 |
| 3137.51 | 63.50 | 19040043.26 | 8184719.68 |
| 3123.34 | 69.72 | 36645311.93 | 30081428.92 |
| 3118.01 | 71.48 | 44067140.21 | 26555313.13 |
| 3108.89 | 79.32 | 5889624.46 | 1439562.41 |
| 3100.69 | 75.26 | 29965185.55 | 32550305.40 |
| 3094.49 | 71.53 | 54313000.91 | 23050752.13 |
| 3084.06 | 66.44 | 18481158.87 | 6713972.52 |
| 3079.03 | 64.80 | 8103891.20 | 4993435.84 |
| 3077.68 | 64.41 | 11687966.61 | 3240628.54 |
| 3072.82 | 65.08 | 20587780.30 | 20124276.56 |
| 3064.29 | 72.58 | 8227078.14 | 15580479.21 |
| 3057.42 | 68.48 | 39718281.86 | 9377053.23 |
| 3051.02 | 69.91 | 7530292.96 | 3831171.52 |
| 3048.24 | 67.97 | 8092333.91 | 5955175.37 |
| 3041.34 | 68.52 | 15661021.41 | 6049547.45 |
| 3035.75 | 67.76 | 15848549.59 | 6976267.39 |
| 3032.56 | 66.99 | 14921658.96 | 12904196.19 |
| 3026.92 | 71.85 | 8727848.47 | 5745223.39 |
| 3020.82 | 74.17 | 27598215.76 | 19115016.02 |
| 3014.15 | 72.52 | 31131098.06 | 20134020.26 |
| 3005.09 | 77.01 | 33070045.94 | 4889595.38 |
| 2997.62 | 74.34 | 19483244.25 | 20797015.78 |
| 2988.71 | 73.64 | 25564484.40 | 29187211.15 |
| 2982.61 | 71.77 | 37910618.24 | 35323615.90 |
| 2977.41 | 70.28 | 10527192.45 | 7420966.69 |
| 2972.29 | 71.06 | 10050763.60 | 15236994.47 |
| 2969.51 | 70.29 | 25443835.77 | 8502071.87 |
| 2955.40 | 74.63 | 27166230.76 | 7637200.34 |
| 2949.88 | 75.15 | 27318687.61 | 9401811.86 |
| 2942.99 | 77.15 | 38585091.13 | 38689402.05 |
| 2934.13 | 84.18 | 5989736.29 | 2052313.64 |
| 2926.12 | 90.00 | 28725293.96 | 42697794.51 |
| 2918.13 | 90.06 | 58237565.60 | 39592431.39 |
| 2906.70 | 98.56 | 54076631.74 | 56295963.75 |
| 2893.07 | 97.81 | 23169511.49 | 15541960.08 |
| 2888.20 | 98.20 | 18723248.42 | 10504463.32 |
| 2884.01 | 97.18 | 22156443.39 | 14028734.39 |
| 2872.07 | 94.48 | 28671914.40 | 38114621.10 |
| 2861.63 | 92.08 | 61409374.66 | 28200336.15 |
| 2853.75 | 90.89 | 10786206.92 | 7692477.52 |
| 2851.21 | 91.74 | 14595119.39 | 8381741.16 |
| 2846.19 | 90.00 | 23273299.55 | 12429068.12 |
| 2835.80 | 95.56 | 19336911.80 | 17001727.45 |
| 2827.86 | 93.55 | 8877100.52 | 9068870.06 |
| 2822.41 | 89.55 | 15835526.79 | 16907163.41 |
| 2819.02 | 87.39 | 19271448.96 | 21210559.56 |
| 2814.81 | 84.28 | 12017987.53 | 9138264.00 |
| 2808.32 | 82.27 | 19684188.80 | 7819937.66 |
| 2805.19 | 81.35 | 15139176.78 | 16312211.05 |
| 2795.32 | 73.80 | 40385980.59 | 3023772.39 |
| 2789.92 | 74.46 | 13714579.43 | 2484950.91 |
| 2783.54 | 76.49 | 13128060.30 | 7235481.09 |
| 2778.50 | 79.56 | 20025295.07 | 17769176.18 |
| 2773.95 | 78.42 | 31978467.52 | 14828688.66 |
| 2768.56 | 82.01 | 13971814.33 | 8845816.58 |
| 2762.30 | 80.67 | 13479776.84 | 6737948.36 |
| 2759.05 | 79.72 | 18220985.01 | 503018.26 |
| 2755.16 | 81.28 | 2669732.32 | 2608631.65 |
| 2744.14 | 86.48 | 66874118.58 | 4951549.34 |
| 2736.14 | 86.97 | 15227619.88 | 23217364.19 |
| 2729.24 | 83.67 | 40047790.36 | 14621368.10 |
| 2721.78 | 86.47 | 32494845.54 | 9861038.35 |
| 2715.02 | 84.99 | 15566052.72 | 8014560.31 |
| 2708.50 | 86.17 | 32274863.45 | 21064023.15 |
| 2703.23 | 89.34 | 17707662.86 | 9238787.31 |
| 2693.93 | 87.00 | 24576409.78 | 14267235.68 |
| 2688.55 | 86.71 | 33478516.12 | 13966490.89 |
| 2682.50 | 85.54 | 7418656.27 | 7893251.31 |
| 2680.13 | 86.01 | 14684559.64 | 5040338.06 |
| 2674.98 | 85.99 | 17270487.71 | 19359469.43 |
| 2671.23 | 84.33 | 26727133.13 | 22398149.03 |
| 2665.67 | 88.83 | 5283789.07 | 3865732.85 |
| 2658.27 | 88.08 | 25273120.09 | 4393075.10 |
| 2653.65 | 89.16 | 21547519.99 | 10611230.68 |
| 2649.35 | 85.70 | 13122469.10 | 8028955.22 |
| 2644.16 | 87.47 | 10781319.39 | 1787055.71 |
| 2640.07 | 88.57 | 11736706.58 | 8250586.67 |
| 2636.65 | 89.85 | 10649457.50 | 6282520.10 |
| 2632.87 | 86.40 | 15703800.97 | 9817664.30 |
| 2626.10 | 90.30 | 19714480.57 | 7192677.53 |
| 2617.47 | 85.43 | 31226146.63 | 12152168.58 |
| 2610.05 | 79.00 | 41708370.08 | 2574178.30 |
| 2600.80 | 83.03 | 36082461.25 | 13946996.68 |
| 2592.59 | 78.32 | 26693374.13 | 16492181.96 |
| 2585.54 | 82.28 | 27921741.92 | 2302322.65 |
| 2578.10 | 81.89 | 15505572.89 | 21037839.04 |
| 2571.80 | 79.51 | 34182656.60 | 14866566.57 |
| 2566.30 | 81.58 | 10833201.94 | 11266461.17 |
| 2560.42 | 82.35 | 34069769.06 | 32034403.31 |
| 2552.22 | 75.43 | 3378399.87 | 2644712.96 |
| 2547.93 | 76.08 | 39196474.09 | 11566189.71 |
| 2538.71 | 75.47 | 10921385.07 | 7323148.51 |
| 2530.88 | 76.21 | 49198701.67 | 495839.33 |
| 2520.43 | 72.41 | 16925388.39 | 4190562.13 |
| 2512.78 | 78.61 | 27034734.49 | 19178045.09 |
| 2507.07 | 76.66 | 12105670.72 | 7318551.29 |
| 2503.62 | 75.64 | 8231720.99 | 4926449.30 |
| 2501.89 | 75.25 | 2695697.87 | 742949.18 |
| 2496.47 | 74.05 | 45085195.33 | 2401171.89 |
| 2486.16 | 71.12 | 11333343.46 | 6507735.71 |
| 2481.80 | 70.62 | 21497226.33 | 978699.11 |
| 2476.14 | 74.41 | 25065131.93 | 2427967.69 |
| 2467.28 | 70.91 | 13698532.63 | 16366245.31 |
| 2464.08 | 69.23 | 19854347.56 | 18973328.27 |
| 2456.24 | 70.64 | 16426566.99 | 16222940.71 |
| 2451.65 | 73.30 | 15780379.69 | 9008397.92 |
| 2447.90 | 72.24 | 17762250.78 | 7401549.72 |
| 2440.34 | 73.88 | 22477712.86 | 13681518.10 |
| 2437.27 | 72.67 | 33473985.92 | 6001138.09 |
| 2430.21 | 70.83 | 9896984.85 | 6482298.70 |
| 2422.80 | 71.42 | 20767328.32 | 12227382.26 |
| 2414.26 | 77.06 | 36194417.10 | 7221990.85 |
| 2410.11 | 76.89 | 13225126.18 | 6849103.58 |
| 2404.84 | 80.56 | 15972011.56 | 15761214.35 |
| 2397.44 | 79.13 | 42447972.69 | 3116361.06 |
| 2389.44 | 81.77 | 12312179.10 | 7824745.27 |
| 2387.23 | 82.96 | 7638563.78 | 3042873.54 |
| 2384.36 | 80.57 | 6792608.82 | 1830983.51 |
| 2377.00 | 81.79 | 19979882.56 | 8784863.39 |
| 2373.87 | 82.50 | 19391076.68 | 9047207.39 |
| 2367.94 | 80.35 | 10842751.05 | 1338523.23 |
| 2358.39 | 84.95 | 67605624.69 | 16696518.60 |
| 2346.18 | 73.79 | 17915481.75 | 6782703.98 |
| 2340.73 | 77.94 | 13405582.97 | 16646151.32 |
| 2338.52 | 77.42 | 20400378.89 | 20392483.90 |
| 2332.74 | 76.88 | 12762647.74 | 8192561.47 |
| 2330.07 | 76.14 | 3695080.60 | 3155341.88 |
| 2329.22 | 75.86 | 5267830.78 | 3066580.05 |
| 2323.74 | 72.19 | 11773784.40 | 16320386.96 |
| 2317.52 | 73.83 | 34610806.46 | 19678295.79 |
| 2311.99 | 73.46 | 38049986.58 | 22383013.79 |
| 2307.60 | 75.80 | 21589527.00 | 2465183.16 |
| 2301.23 | 77.84 | 9510428.47 | 8605995.62 |
| 2296.66 | 76.30 | 24513429.85 | 3249221.38 |
| 2292.96 | 75.74 | 3722069.07 | 3539327.35 |
| 2287.59 | 77.52 | 33697641.24 | 27030677.63 |
| 2279.40 | 82.47 | 33667781.63 | 25102629.42 |
| 2276.00 | 82.95 | 51327515.38 | 20485990.22 |
| 2263.79 | 74.98 | 19259319.45 | 19184327.67 |
| 2262.77 | 75.04 | 34737064.85 | 9498311.75 |
| 2251.65 | 77.49 | 22268464.86 | 16528203.81 |
| 2249.40 | 77.60 | 30879684.57 | 18577805.62 |
| 2239.40 | 78.95 | 12661031.88 | 7926297.90 |
| 2234.25 | 77.38 | 21016522.69 | 10555362.43 |
| 2230.89 | 76.54 | 28617627.12 | 81636.22 |
| 2224.33 | 76.96 | 13832527.48 | 7198557.38 |
| 2222.02 | 76.31 | 12418154.19 | 12094252.83 |
| 2216.52 | 78.35 | 16600502.80 | 9176382.93 |
| 2213.54 | 79.17 | 16931528.32 | 11801163.54 |
| 2207.17 | 77.82 | 26738129.67 | 10901707.68 |
| 2204.60 | 79.18 | 21147827.70 | 21699405.87 |
| 2198.23 | 75.24 | 16466096.25 | 6976682.27 |
| 2192.99 | 71.51 | 8450148.18 | 6644247.22 |
| 2189.93 | 72.42 | 15984063.74 | 15914916.09 |
| 2186.73 | 71.56 | 23277490.55 | 17871975.88 |
| 2180.00 | 75.57 | 17250213.66 | 9016909.20 |
| 2174.61 | 78.38 | 19066625.56 | 18471707.30 |
| 2171.39 | 81.13 | 16727577.51 | 14878434.86 |
| 2167.57 | 81.79 | 17486637.04 | 16089300.86 |
| 2160.34 | 81.30 | 55117901.61 | 60827538.28 |
| 2149.22 | 90.56 | 7330904.48 | 3880842.06 |
| 2148.05 | 90.60 | 10061016.94 | 1428645.33 |
| 2142.13 | 90.49 | 17187131.15 | 15201450.97 |
| 2138.95 | 89.15 | 27732772.22 | 12816866.81 |
| 2132.64 | 94.07 | 23160451.34 | 7451209.21 |
| 2129.20 | 92.91 | 19895001.30 | 15037326.02 |
| 2124.32 | 93.49 | 21647446.88 | 5014265.55 |
| 2118.49 | 90.68 | 31583760.09 | 39696.62 |
| 2112.65 | 93.18 | 23618680.16 | 7816955.71 |
| 2103.87 | 88.87 | 19067735.65 | 12282181.23 |
| 2101.18 | 88.82 | 27141754.05 | 5110877.70 |
| 2093.21 | 85.81 | 30968672.45 | 25786526.05 |
| 2087.98 | 81.89 | 7652250.80 | 2778589.51 |
| 2084.72 | 84.52 | 30340667.85 | 26243240.46 |
| 2079.30 | 88.75 | 23336380.16 | 10191775.32 |
| 2074.44 | 87.27 | 4413018.54 | 975274.21 |
| 2072.69 | 87.67 | 3599873.96 | 2013970.86 |
| 2070.55 | 88.16 | 17342208.42 | 16992808.57 |
| 2066.72 | 90.28 | 2780537.52 | 1703714.49 |
| 2062.82 | 88.08 | 9287962.27 | 1210143.08 |
| 2060.27 | 88.30 | 15994010.06 | 14591780.36 |
| 2055.54 | 84.71 | 7328859.22 | 2561419.94 |
| 2048.65 | 80.25 | 31398129.93 | 22701765.90 |
| 2045.69 | 79.37 | 12548381.88 | 2322244.68 |
| 2039.23 | 78.24 | 45574115.58 | 25496143.29 |
| 2027.74 | 74.49 | 29120916.22 | 18892121.62 |
| 2023.66 | 74.48 | 30730437.22 | 20507268.28 |
| 2016.77 | 73.36 | 16975844.49 | 8032399.88 |
| 2014.17 | 72.69 | 20494142.08 | 1388076.84 |
| 2008.18 | 69.77 | 14062256.07 | 1918569.47 |
| 2004.98 | 69.01 | 7766693.94 | 1726234.57 |
| 2001.38 | 67.17 | 17555377.17 | 13683669.28 |
| 1996.53 | 67.96 | 15183356.16 | 17159307.34 |
| 1994.08 | 68.55 | 20721744.25 | 18039134.48 |
| 1984.69 | 70.97 | 32665114.41 | 6245320.60 |
| 1978.14 | 73.80 | 28168134.15 | 306843.58 |
| 1972.58 | 71.67 | 9983946.70 | 3454559.41 |
| 1969.84 | 71.69 | 17353347.62 | 13486732.22 |
| 1962.66 | 71.16 | 31890470.65 | 28696808.33 |
| 1955.18 | 66.40 | 19687405.65 | 17400459.78 |
| 1952.66 | 65.97 | 29178846.18 | 18940406.32 |
| 1949.44 | 64.51 | 16979591.33 | 10075425.44 |
| 1944.16 | 66.79 | 15523473.36 | 6258297.22 |
| 1940.78 | 65.95 | 5705294.62 | 5019942.34 |
| 1939.08 | 66.51 | 4150369.47 | 1204834.73 |
| 1934.52 | 65.88 | 50279169.44 | 7135733.27 |
| 1927.94 | 68.30 | 19603220.48 | 16744393.40 |
| 1923.21 | 72.87 | 5881498.34 | 202589.62 |
| 1920.20 | 74.15 | 9083859.30 | 4310570.91 |
| 1917.28 | 72.06 | 17377661.61 | 2139713.46 |
| 1911.14 | 76.77 | 27008387.97 | 22092224.62 |
| 1905.91 | 76.89 | 31224136.45 | 32602387.72 |
| 1900.23 | 78.73 | 27662460.12 | 14202731.43 |
| 1895.79 | 80.81 | 20288691.39 | 18805809.02 |
| 1890.31 | 80.58 | 18507274.18 | 11899128.80 |
| 1884.50 | 82.79 | 23273916.64 | 14086832.57 |
| 1881.14 | 84.43 | 21021152.50 | 16986700.93 |
| 1876.69 | 82.45 | 15323751.91 | 9112165.05 |
| 1872.80 | 84.84 | 29895119.73 | 13106557.12 |
| 1866.47 | 83.47 | 14095096.03 | 8664460.73 |
| 1861.14 | 84.13 | 18284073.95 | 11297956.75 |
| 1859.15 | 84.16 | 21472881.03 | 7119960.92 |
| 1854.31 | 83.13 | 18717665.33 | 12336629.53 |
| 1852.64 | 82.86 | 24657935.23 | 12935523.67 |
| 1847.60 | 81.29 | 15686493.65 | 9901435.51 |
| 1842.79 | 77.92 | 10704277.55 | 10762227.08 |
| 1837.79 | 79.10 | 27527289.47 | 12018037.66 |
| 1834.07 | 80.25 | 7654986.21 | 6806629.36 |
| 1830.92 | 81.94 | 17281665.93 | 7176581.34 |
| 1824.93 | 83.27 | 25318012.89 | 27057360.22 |
| 1820.45 | 83.73 | 52492545.65 | 13559729.17 |
| 1813.07 | 80.85 | 14872603.40 | 1901051.15 |
| 1806.94 | 78.07 | 45318420.43 | 16101971.73 |
| 1801.94 | 78.70 | 7913796.27 | 2410831.89 |
| 1799.52 | 78.44 | 13142802.41 | 4186108.74 |
| 1795.30 | 78.75 | 29968728.66 | 21196423.90 |
| 1790.63 | 75.78 | 41172977.04 | 19064114.05 |
| 1779.38 | 67.93 | 40688660.05 | 46508273.27 |
| 1776.74 | 66.56 | 2962514.49 | 724516.02 |
| 1774.33 | 65.29 | 18187824.69 | 17753519.51 |
| 1770.69 | 63.23 | 31793423.47 | 28319595.26 |
| 1762.17 | 56.32 | 13179813.83 | 14877684.69 |
| 1758.67 | 57.13 | 66708798.32 | 38215140.29 |
| 1751.64 | 57.49 | 34429398.99 | 26114382.08 |
| 1747.24 | 54.79 | 36285932.62 | 37703784.71 |
| 1739.88 | 61.78 | 6964022.43 | 4600629.08 |
| 1737.19 | 62.29 | 7697357.10 | 4052505.50 |
| 1734.43 | 60.71 | 21985181.37 | 12736599.05 |
| 1729.02 | 57.69 | 23882756.92 | 8858907.53 |
| 1721.57 | 52.14 | 50870806.45 | 49797820.46 |
| 1712.46 | 45.10 | 24263580.92 | 15186916.98 |
| 1706.26 | 45.02 | 30511773.52 | 15564404.48 |
| 1700.78 | 43.54 | 28016393.48 | 14108489.57 |
| 1691.13 | 36.63 | 35108988.32 | 35671765.16 |
| 1684.97 | 33.17 | 32720714.56 | 29517546.61 |
| 1681.39 | 31.64 | 40372346.30 | 37580574.64 |
| 1677.66 | 31.03 | 10083221.90 | 9842889.10 |
| 1674.82 | 32.41 | 14077675.84 | 3264717.85 |
| 1670.18 | 29.22 | 38364245.24 | 28421815.74 |
| 1661.53 | 25.56 | 35186159.98 | 12526902.94 |
| 1657.27 | 22.51 | 21962778.79 | 22758040.35 |
| 1646.81 | 20.91 | 39561118.20 | 9611280.36 |
| 1639.69 | 17.75 | 70362502.17 | 19800253.72 |
| 1623.91 | 23.94 | 71989697.44 | 77097377.73 |
| 1616.75 | 28.13 | 18078945.11 | 6801902.60 |
| 1613.73 | 28.51 | 12601310.15 | 11732186.68 |
| 1608.26 | 29.70 | 22665641.25 | 7346444.49 |
| 1603.34 | 28.69 | 30073688.45 | 5063255.00 |
| 1600.06 | 28.50 | 19817910.18 | 16742721.02 |
| 1594.77 | 25.19 | 29767782.34 | 12651483.21 |
| 1591.46 | 26.19 | 25160053.37 | 14053755.90 |
| 1587.81 | 23.15 | 32740069.22 | 7752483.37 |
| 1577.50 | 27.34 | 44155964.40 | 22113236.10 |
| 1574.17 | 29.27 | 2618412.92 | 2739445.25 |
| 1572.48 | 29.96 | 8283974.26 | 435382.92 |
| 1567.70 | 30.87 | 35268646.70 | 27272113.56 |
| 1563.74 | 32.74 | 57311974.32 | 16437414.62 |
| 1554.30 | 25.87 | 38180847.23 | 9009048.90 |
| 1544.06 | 26.58 | 40809046.72 | 10263841.48 |
| 1539.87 | 26.59 | 31718484.43 | 17607978.22 |
| 1535.18 | 28.11 | 46717446.35 | 33662799.97 |
| 1529.10 | 31.37 | 15159581.47 | 22717428.35 |
| 1527.41 | 31.23 | 44339825.44 | 5458202.03 |
| 1518.83 | 32.72 | 23140434.72 | 11530542.73 |
| 1513.63 | 34.93 | 22581528.40 | 19821543.03 |
| 1510.00 | 36.67 | 13460381.12 | 3185724.52 |
| 1507.90 | 36.01 | 22338977.67 | 14662503.91 |
| 1504.83 | 38.62 | 37857106.76 | 7398153.07 |
| 1498.30 | 34.99 | 23455026.94 | 14999137.06 |
| 1471.78 | 10.77 | 154656464.21 | 139271192.43 |
| 1458.56 | 0.93 | 90080868.28 | 40953029.86 |
| 1448.81 | 0.79 | 40436778.17 | 10521402.98 |
| 1446.08 | 1.39 | 5336848.90 | 4681017.31 |
| 1442.99 | 3.38 | 29626710.32 | 31254997.18 |
| 1436.03 | 5.97 | 72212769.22 | 12704723.32 |
| 1425.05 | 11.07 | 48305773.28 | 25252135.06 |
| 1420.39 | 10.81 | 44522025.94 | 45324620.71 |
| 1414.14 | 10.10 | 38770175.08 | 58152491.82 |
| 1407.93 | 4.65 | 77562603.59 | 55895325.59 |
| 1403.12 | 1.24 | 35073914.18 | 12491904.33 |
| 1388.74 | 4.16 | 89732035.58 | 11915766.84 |
| 1381.90 | 1.94 | 36924967.60 | 30283407.95 |
| 1380.40 | 1.31 | 34664243.09 | 35268757.89 |
| 1374.98 | 6.55 | 3189606.98 | 756150.71 |
| 1369.88 | 10.39 | 32520115.84 | 27236740.14 |
| 1366.68 | 11.29 | 43834473.71 | 30662758.00 |
| 1358.08 | 7.28 | 59090476.39 | 36152386.44 |
| 1351.25 | 8.21 | 74618701.10 | 37123567.08 |
| 1344.03 | 3.30 | 26923014.64 | 27446679.17 |
| 1338.35 | 3.56 | 45757983.03 | 3986455.48 |
| 1333.94 | 5.01 | 39154116.34 | 8667291.47 |
| 1327.25 | 3.25 | 35680822.22 | 3853203.88 |
| 1323.35 | 2.86 | 37751869.64 | 2452787.33 |
| 1320.95 | 0.49 | 34482975.59 | 1749809.66 |
| 1316.80 | 1.37 | 10396138.14 | 6810361.34 |
| 1312.93 | 1.86 | 53676390.81 | 25869702.20 |
| 1306.92 | 6.48 | 47506102.23 | 1971607.94 |
| 1300.99 | 6.45 | 28227492.69 | 9821166.98 |
| 1299.72 | 6.83 | 31811940.40 | 8197514.94 |
| 1294.91 | 7.21 | 20838316.31 | 11141120.38 |
| 1289.17 | 6.20 | 42867890.50 | 18552533.14 |
| 1285.04 | 4.27 | 49059615.19 | 14248882.44 |
| 1282.00 | 2.75 | 41860201.65 | 32757873.62 |
| 1275.82 | 2.67 | 35067732.33 | 31922106.43 |
| 1273.99 | 2.30 | 47317853.29 | 41105587.82 |
| 1268.37 | 4.66 | 19566412.02 | 16320587.04 |
| 1259.12 | 8.59 | 109923932.10 | 18133902.92 |
| 1252.95 | 6.76 | 54738932.66 | 60212880.81 |
| 1247.56 | 1.60 | 14417418.59 | 13735264.29 |
| 1242.22 | 2.95 | 41553114.16 | 28582701.00 |
| 1239.26 | 2.52 | 57398324.08 | 18729637.28 |
| 1234.34 | 4.59 | 53774436.49 | 34118311.02 |
| 1228.21 | 0.24 | 83321749.38 | 12519601.00 |
| 1221.31 | 1.67 | 63564527.51 | 31521726.74 |
| 1216.96 | 3.52 | 60178620.27 | 24339697.06 |
| 1210.51 | 2.35 | 36346810.13 | 7334799.47 |
| 1208.30 | 1.32 | 36004719.04 | 13099228.96 |
| 1205.31 | 3.28 | 21179465.98 | 9588045.22 |
| 1200.84 | 6.47 | 61510491.41 | 32154185.38 |

1. An aqueous extract useful notably for the regeneration of human or mammalian animal cells, comprising an aqueous extract free of any insoluble solid debris of ground organisms of the Platyhelminthes type having a regeneration capacity, said organisms being aquatic flatworms of the planarian type which are non-parasitic and non-pathogenic in humans or mammalian animals, said aqueous extract containing at least the intracellular components of the cells of said organisms of a cell lysate of said cells of said whole organisms.

2. The aqueous extract as claimed in claim 1 characterized in that said organisms are of the species Schmidtea mediterranea.

3. The aqueous extract as claimed in claim 1 characterized in that said aqueous extract of ground material comprises a centrifugation supernatant of said ground material.

4. The aqueous extract as claimed in claim 1 characterized in that said aqueous extract of ground material further comprises a cell culture medium, preferably a culture medium capable of culturing human or animal cells to be regenerated.

5. The aqueous extract as claimed in claim 1 characterized in that said aqueous extract of ground material comprises the grinding of 50 to 200 mg worms per mL aqueous solution; preferably 75 to 150 mg worms per mL aqueous solution, said aqueous extract of ground material being further diluted preferably more than 1:5 and less than 1:100, preferably diluted between 1:10 and 1:50.

6. The aqueous extract as claimed in claim 5 characterized in that said aqueous extract of ground material comprises the grinding of 110 mg worms per mL aqueous solution; said aqueous extract of ground material being further diluted 1:20.

7. The aqueous extract as claimed in claim 1 characterized in that a spectrum of said preparation by MALDI TOF mass spectrometry comprises the 16 characteristic majority peaks mentioned in the following Table 1, wherein the individual characteristic peaks are numbered in the first column, and the following parameters have the following meanings:
M/z: ratios (mass in daltons/charge) on the x-axis of the different characteristic peaks of the spectrum,
S/N: ratios (peak intensity/background noise intensity) of the different characteristic peaks of the spectrum,
TABLE 1

| | | M/Z | | S/N |
|---|---|---|---|---|
| | | standard | | standard |
| | M/Z | deviation | S/N | deviation |
| 1 | 8618.401 | 118.031 | 10852424.610 | 11358052.088 |
| 2 | 8270.340 | 143.008 | 4620996.274 | 5564954.057 |
| 3 | 7696.982 | 133.512 | 5240359.923 | 7010776.551 |
| 4 | 7236.251 | 151.442 | 8121705.361 | 11556323.264 |
| 5 | 6803.343 | 116.896 | 10708147.034 | 18373045.879 |
| 6 | 6261.480 | 163.376 | 11319617.938 | 13604702.931 |
| 7 | 5790.573 | 116.345 | 21402383.693 | 37624416.818 |
| 8 | 5330.950 | 180.245 | 23085485.049 | 21438811.717 |
| 9 | 4787.963 | 120.365 | 22731248.668 | 23171439.149 |
| 10 | 4298.636 | 175.285 | 24470357.501 | 21953618.321 |
| 11 | 3793.559 | 113.425 | 21664847.045 | 17673326.341 |
| 12 | 3282.861 | 172.508 | 24634530.736 | 27003268.283 |
| 13 | 2792.441 | 122.205 | 22551669.576 | 22432014.766 |
| 14 | 2294.281 | 174.311 | 20589324.299 | 17568023.101 |
| 15 | 1807.544 | 115.976 | 19890585.114 | 18182405.582 |
| 16 | 1395.545 | 119.208 | 33019669.177 | 29193485.572. |

8. A lyophilisate of an aqueous extract of ground material as claimed in claim 1.

9. A process for preparing an aqueous extract as claimed in claim 1 characterized in that the steps are carried out in which:
a) said living organisms, preferably washed with water and not fed for at least one week, are placed in an aqueous liquid medium containing preferably a cell culture medium;
b) the aqueous preparation obtained in step a) is ground until lysis of the cells of said organisms is obtained, preferably the ground material debris being between 5 and 10 μm in size,
c) insoluble debris is removed by centrifugation; and the supernatant is collected, and
d) preferably, the centrifugation supernatant is diluted in an aqueous solution more than 1:5 and less than 1:100, preferably diluted between 1:10 and 1:50, said solution comprising preferably still components of a cell culture medium capable of culturing human or animal cells to be regenerated.

10. Use of an aqueous extract or lyophilisate as claimed in claim 1 for the manufacture of a composition useful for regenerating human or mammalian animal cells.

11. Use of an aqueous extract or lyophilisate as claimed in claim 10 for the manufacture of a composition useful for regenerating human or mammalian animal stem cells.

12. Use of an aqueous extract or lyophilisate as claimed in claim 10 in combination with suitable carriers and/or excipients for the manufacture of a food, cosmetic or dermatological composition.

13. A cosmetic composition with an anti-ageing effect on the skin comprising an aqueous or lyophilized extract as claimed in claim 1 and excipients suitable for topical application to the skin of said composition in the form of gel, lotion, cream, ointment or soap.

14. Use of an aqueous extract or lyophilisate as claimed in claim 1 for regenerating human or mammalian animal cells.

15. Use of an aqueous extract or lyophilisate as claimed in claim 10 for regenerating human or mammalian animal stem cells.
